(19) United States
(12) Patent Application Publication
(10) Pub. No.: US 2007/0027401 A1
(43) Pub. Date: Feb. 1, 2007

(54) Body composition analyzer

(75) Inventors: Miyuki Shimomura, Tokyo (JP); Wataru Uchikoshi, Saitama (JP); Soji Kurata, Saitama (JP)

Correspondence Address: MCDERMOTT WILL & EMERY LLP, 600 13TH STREET, N.W., WASHINGTON, DC, 20005-3096, US

(73) Assignee: TANITA CORPORATION, Tokyo (JP)

(21) Appl. No.: 10/545,325

(22) PCT Filed: Aug. 26, 2004
(86) PCT No.: PCT/IB04/03006

§ 371 Date: Aug. 12, 2005

Related U.S. Application Data
(60) Provisional application No. 60/498,305, filed on Aug. 28, 2003.
(60) Provisional application No. 60/501,096, filed on Sep. 9, 2003.

(51) Int. Cl. A61B 5/05

(52) U.S. Cl. 600/547

(57) ABSTRACT

A body composition analyzer using a bioelectric-impedance method includes a display unit for displaying measurement results including a weight and a body fat ratio, and a processor responsive to a user's input for causing the display means to display a part of the measurement results. In one embodiment, the display unit displays at least the body fat ratio, and the measurement result caused to be displayed by the processor is one of basal metabolism data, visceral fat data, muscle mass data, and bone mass data. The display unit displays an item selected from among the basal metabolism data, the visceral fat data, the muscle mass data, and the bone mass data if the item is designated by the user while the body fat ratio is being displayed, and then displays another item if that item is subsequently designated by the user. Thus, a main measurement result and a desired secondary measurement result can be viewed without waiting, and two or more desired secondary measurement results can be selected and viewed.

### FIELD OF THE INVENTION

The present invention relates to a device for measuring the body composition of an organism using a bioelectric-impedance method.

### BACKGROUND OF THE INVENTION

Devices for determining body fat ratio, basal metabolism rate, etc., by measuring bioelectric impedance are known in the art. In these devices, when the measurement is finished, measurement results are sequentially displayed, alternatively displayed, or printed out with a printer.

When a plurality of measurement results are sequentially displayed, in order to check a desired measurement result, a user must wait until it appears on the display. In addition, if the user fails to check the desired measurement result, the measurement must be performed again.

In devices in which the sequential display is repeated to prevent oversight, the user must wait for the next cycle if he or she fails to check the desired measurement result.

When the measurement results are alternatively displayed, a main measurement result, for example, a fat ratio, and secondary measurement results, for example, a basal metabolism rate and a visceral fat area, are displayed alternately. More specifically, the fat ratio is displayed first, then the basal metabolism rate, then the fat ratio again, and then the visceral fat area. Accordingly, the display constantly changes and the measurement results cannot be easily viewed and recognized by the user.

### SUMMARY OF THE INVENTION

The present invention solves the above-described problems of oversight and ease of viewing.

According to the present invention, a body composition analyzer using a bioelectric-impedance method includes a display unit for displaying measurement results including a weight and a body fat ratio and a processor responsive to a user's input for causing the display means to display a part of the measurement results.

In addition, according to the present invention, the display unit displays at least the body fat ratio, and the measurement result caused to be displayed by the processor is one of basal metabolism data, visceral fat data, muscle mass data, and bone mass data.

In addition, according to the present invention, the display unit displays an item selected from among the basal metabolism data, the visceral fat data, the muscle mass data, and the bone mass data if the item is designated by the user while the body fat ratio is being displayed, and then displays another item if that item is subsequently designated by the user.

Since the body composition analyzer of the present invention has a display unit for displaying measurement results including weight and body fat ratio and a processor for causing the display means to display a part of the measurement results, a main measurement result and a desired secondary measurement result can be viewed without waiting.

In addition, since the display unit of the present invention displays at least the body fat ratio and the measurement result designated by the user is one of basal metabolism data, visceral fat data, muscle mass data, and bone mass data, various measurement results can be selected.

In addition, since the inventive display unit displays an item selected from among the basal metabolism data, the visceral fat data, the muscle mass data, and the bone mass data if the item is designated by the user while the body fat ratio is being displayed and then displays another item if the other item is subsequently designated by the user, two or more desired secondary measurement results can be selected and viewed.

### BRIEF DESCRIPTION OF THE DRAWINGS

FIG. 1 is a perspective view of a body composition analyzer according to an embodiment of the present invention.

FIG. 2 is a diagram showing the detailed structure of the input portion 6 shown in FIG. 1.

FIG. 3 is a block diagram of a body composition analyzer according to an embodiment of the present invention.

FIG. 4 is a flowchart illustrating the operation of a body composition analyzer according to an embodiment of the present invention.

FIGS. 5a and 5b illustrate a display of body weight and body fat percentage, respectively, in a body composition analyzer according to an embodiment of the present invention.

FIG. 6 illustrates a display of basal metabolism in a body composition analyzer according to an embodiment of the present invention.

FIG. 7 illustrates a display of visceral fat level in a body composition analyzer according to an embodiment of the present invention.

FIGS. 8a and 8b illustrate a display of muscle mass and muscle mass score, respectively, in a body composition analyzer according to an embodiment of the present invention.

FIG. 9 illustrates a display of estimated bone mass in a body composition analyzer according to an embodiment of the present invention.

### DETAILED DESCRIPTION OF THE INVENTION

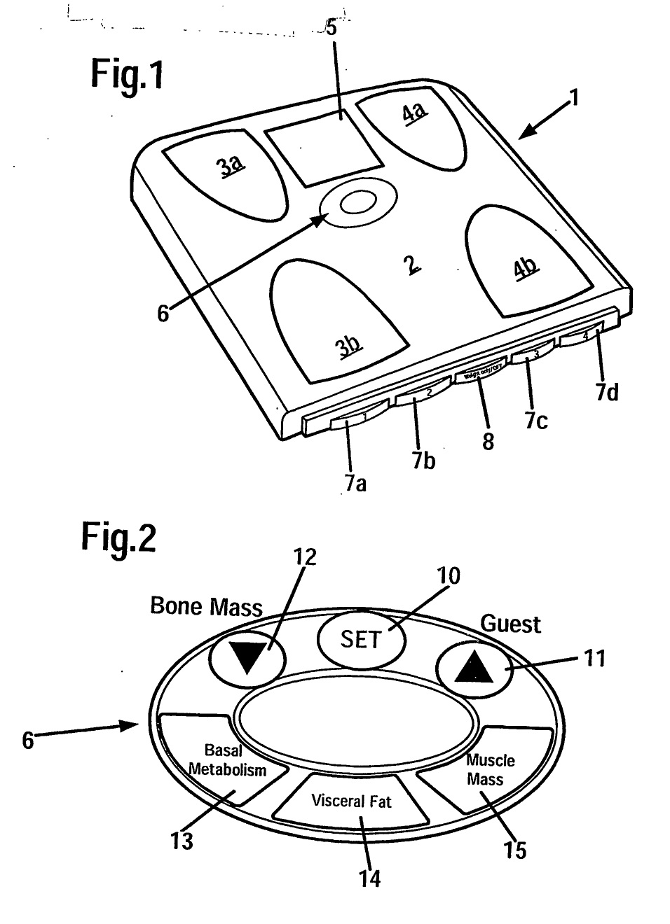

FIG. 1 is a perspective view of a body composition analyzer 1 according to an embodiment of the present invention. The mechanical structure of the body composition analyzer 1 is similar to that of known body fat analyzers, and a conventionaltype weighing scale is included in the body composition analyzer 1. Similar to known body fat analyzers, electrodes 3a, 3b, 4a, and 4b for contacting the soles of the right and left feet of a subject (i.e., a user) are provided on a platform 2 on which the subject stands to measure weight. A display 5 for displaying measurement results and an input portion 6 for inputting various data or designating an item to be displayed are provided on the top face of the platform 2. In addition, four personal keys 7a, 7b, 7c, and 7d for reading out physical data, such as height and age, of respective subjects and a weight only/OFF key 8 for measuring only weight or turning off the power are provided on the front face of the platform 2.

FIG. 2 is a diagram showing the detailed structure of the input portion 6 shown in FIG. 1. A setting key 10 for setting an input mode for inputting the physical data of the subject, an up key 11 for increasing a data value in the input mode, and a down key 12 for reducing the data value in the input mode are provided in an upper area of the input portion 6. In addition, a basal metabolism key 13, a visceral fat key 14, and a muscle key 15 for displaying basal metabolism data, visceral fat data, and muscle data, respectively, which are secondary measurement results, are provided in a lower area of the input portion 6. The down key 12 also functions as a bone mass key for displaying bone mass data, which is also a secondary measurement result. In addition, the up key 11 also functions as a guest key used instead of the four personal keys 7a to 7d (for memorizing and reading out the physical data of the respective subjects) when a one-time user measures body composition.

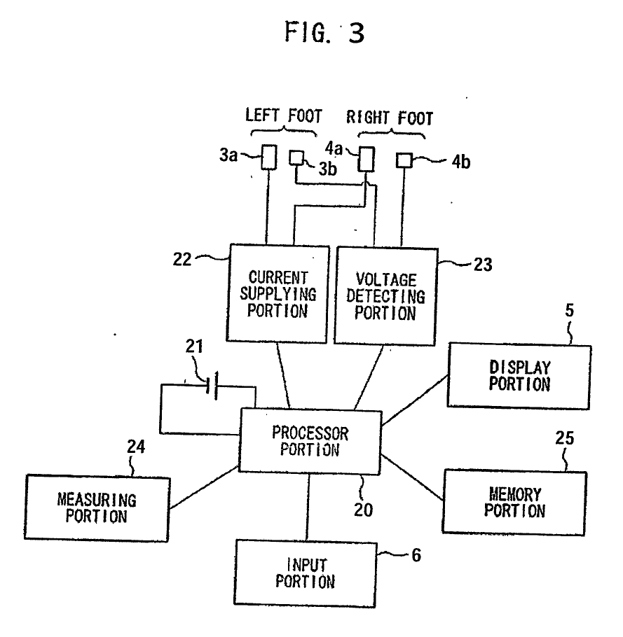

Next, a control block diagram of the body composition analyzer 1 will be described below with reference to FIG. 3. FIG. 3 displays an electrical block diagram of the analyzer 1 shown in FIG. 1. The reference numerals used in FIG. 3 represent the same features shown in FIGS. 1 and 2. A processor portion 20, which processes a variety of data, is connected with a battery 21. It should be noted that the processor portion 20 can be supplied with power via other sources of power, as is known in the art.

A high frequency current supplying portion 22 is connected with the processor portion 20, and the current supplying portion 22 is also connected with the electrodes 3a and 4a. Furthermore, a voltage detecting portion 23 is connected with the processor portion 20, and the voltage detecting portion 23 is also connected with the electrodes 3b and 4b. Still further, the processor portion 20 is connected to a measuring portion 24 for determining body weight, the input portion 6, display 5, and a memory portion 25 to memorize data for processing.

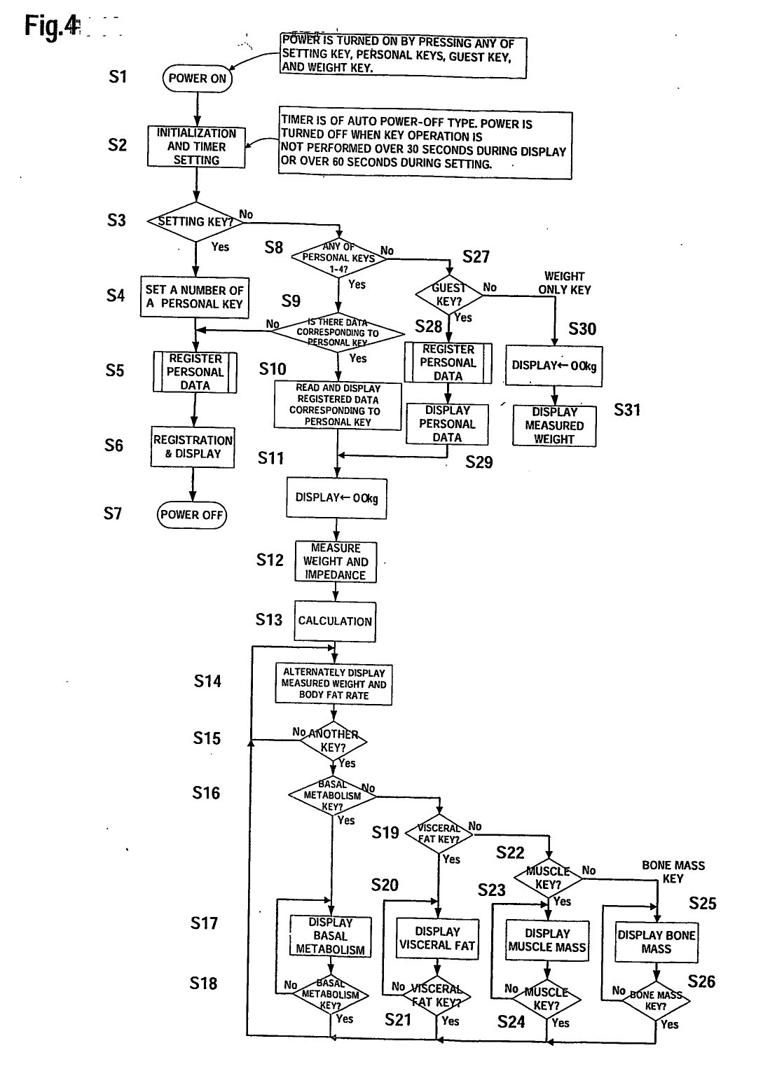

Next, the operation of the body composition analyzer shown in FIGS. 1 to 3 will be described below with reference to a flowchart shown in FIG. 4. First, when any one of the setting key 10, the personal keys 7a to 7d, the guest key 11, and the weight only/OFF key 8 is pressed, the power turns on in Step 1. When the power is turned on, a flag and the like of the processor portion 20 are initialized and an automatic power-off timer is set in Step 2. This timer causes the power to be forcibly turned off if a key operation is not performed for over 60 seconds during setting operations, or for over 30 seconds while measurement or display is being performed.

Next, in Step 3, it is determined whether or not the key pressed in Step 1 is the setting key 10. The result is determined as “Yes” if the pressed key is the setting key 10, and the process proceeds to Step 4. In Step 4, a key for which data is to be registered is selected from among the personal keys 7a to 7d using the up key 11 or the down key 12 of the input portion 6. More specifically, when the key 11 or the key 12 is pressed once, one of the numbers 1 to 4 is shown on the display 5. The displayed number changes when the key 11 or the key 12 is pressed repeatedly, and when the setting key 10 is pressed while a desired number, for example, “1”, is displayed, the process proceeds to Step 5, where personal data is registered. In the registration of the personal data, the subject's age, sex, and height are input. Then, the process proceeds to Step 6 when the setting key 10 is pressed again, and the number of the personal key, the age, the sex, and the height are stored in the memory portion 25 and displayed on the display 5 at the same time. The registration of the personal data is performed similarly to conventional body fat analyzers, and detailed explanations thereof are thus omitted. The automatic power-off timer set in Step 2 runs out during this display, and the power is turned off in Step 7.

If it is determined that the pressed key is not the setting key 10 in Step 3, the process proceeds to Step 8 and it is determined whether or not the pressed key is one of the personal keys 7a to 7d. The result is determined as “Yes” if the pressed key is one of the personal keys 7a to 7d, and the process proceeds to Step 9. In Step 9, it is determined which one of the personal keys 7a to 7d corresponding to the numbers 1 to 4, respectively, was pressed and whether or not personal data is registered for that key. The result is determined as “No” if no data is registered for that key, and the process returns to Step 5 for registering the personal data. If the personal data is registered, the registered data is read out from the memory portion 25 and is displayed on the display 5 (Step 10). More specifically, the number of the personal key, the age, the sex, and the height are sequentially displayed, and then “00 kg” is displayed as the weight in Step 11.

After “00 kg” is displayed, the subject gets on the platform 2 of the body composition analyzer 1 such that his or her soles come into contact with the electrodes 3a, 3b, 4a, and 4b. First, the weight is measured in Step 12, and the weight data is transmitted to the processor portion 20 from the measuring portion 24. Then, the current supplying portion 22 applies a current between the electrodes 3a and 4a which are in contact with the toes, and the voltage detecting portion 23 measures a voltage via the electrodes 3b and 4b which are in contact with the heels. The processor portion 20 calculates the impedance of the subject on the basis of the applied current and the measured voltage in a conventional manner.

Next, in Step 13, a body fat ratio, a basal metabolism rate, a result of evaluation of the basal metabolism rate, a level of visceral fat area, a result of evaluation of the visceral fat area, a muscle mass, a result of evaluation of the muscle mass, and a bone mass are obtained by calculation based on the personal data, the weight, and the impedance. A method for calculating the body fat ratio is already known in the art, and detailed explanations thereof are thus omitted. The basal metabolism rate and the evaluation thereof are performed using techniques described in U.S. Pat. Nos. 6,480,736 and 6,477,409, the entire disclosures of which are hereby incorporated by reference herein. The visceral fat is determined using a technique described in United States Published Patent Application No. 2003/0013982, the entire disclosure of which is hereby incorporated by reference herein. The muscle mass is determined using a technique described in Japanese Patent Application No. 2003-146842, the entire disclosure of which is hereby incorporated by reference herein. The bone mass is determined using a technique described in Japanese Laid-Open Patent Publication No. 2002-65679, the entire disclosure of which is hereby incorporated by reference herein.

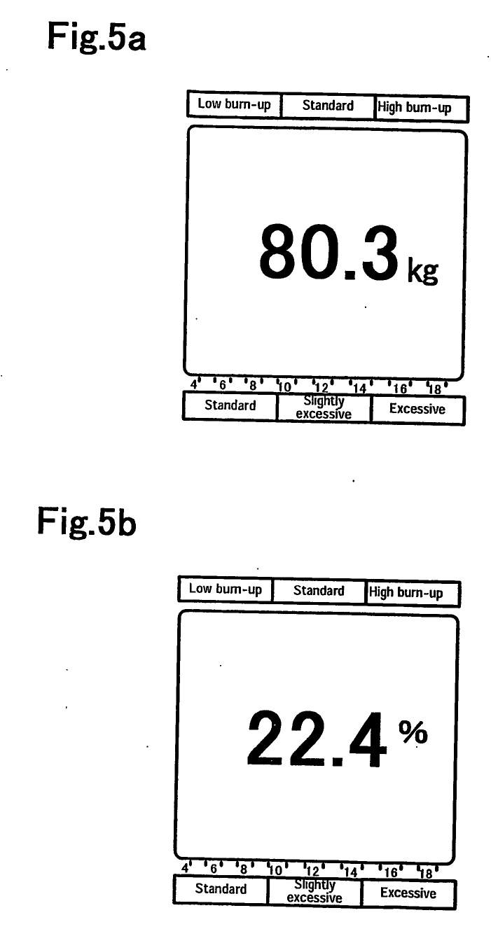

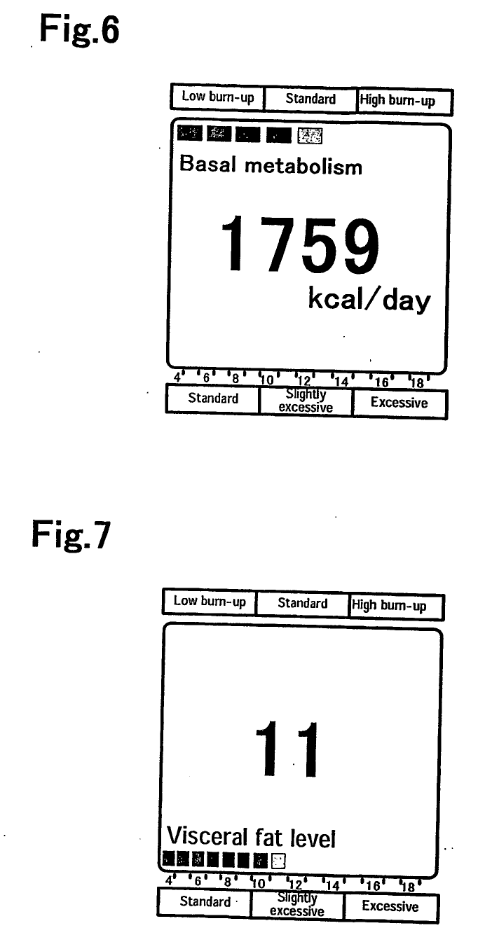

In Step 14, the weight and the body fat ratio, which are the main display items, are alternately displayed as shown in FIGS. 5a and 5b, each item appearing for 3 seconds 3 times. During this 18-second display period, if one of the basal metabolism key 13, the visceral fat key 14, the muscle key 15, and the down key 12 for displaying the bone mass is pressed, the result of Step 15 is determined as “Yes” and the process proceeds to Step 16. If the pressed key is the basal metabolism key 13, the result of Step 16 is determined as “Yes” and the basal metabolism rate and the evaluation result thereof visualized as a bar graph are displayed on the display 5, as shown in FIG. 6.

The basal metabolism rate is evaluated on whether the subject has a body with tendency to be fat or “low burn-up” or a body with a tendency not to be fat or “high burn-up”, and the evaluation result is displayed as the result of calculation relative to the standard. If the basal metabolism key 13 is pressed again while the basal metabolism data is being displayed, the result of Step 18 is determined as “Yes”. Accordingly, the process returns to Step 14 and the display of the main display items, that is, the weight and the body fat ratio, is performed again.

If it is determined that the pressed key is not the basal metabolism key 13 in Step 16, the process proceeds to Step 19 and determines whether or not the pressed key is the visceral fat key 14. If the pressed key is the visceral fat key 14, the result of Step 19 is determined as “Yes” and the process proceeds to Step 20. In Step 20, an integer corresponding to 1/10 of the calculated visceral fat area (cm²) and a bar graph showing the evaluation result of the visceral fat area according to medical associations or the like are displayed, as shown in FIG. 7. In the example shown in FIG. 7, the visceral fat area is 110 cm² and the visceral fat level is determined as 11, which is evaluated as slightly excessive. If the visceral fat key 14 is pressed again while the visceral fat data is being displayed, the result of Step 21 is determined as “Yes”. Accordingly, the process returns to Step 14 and the main display items, the weight and the body fat ratio, are displayed again.

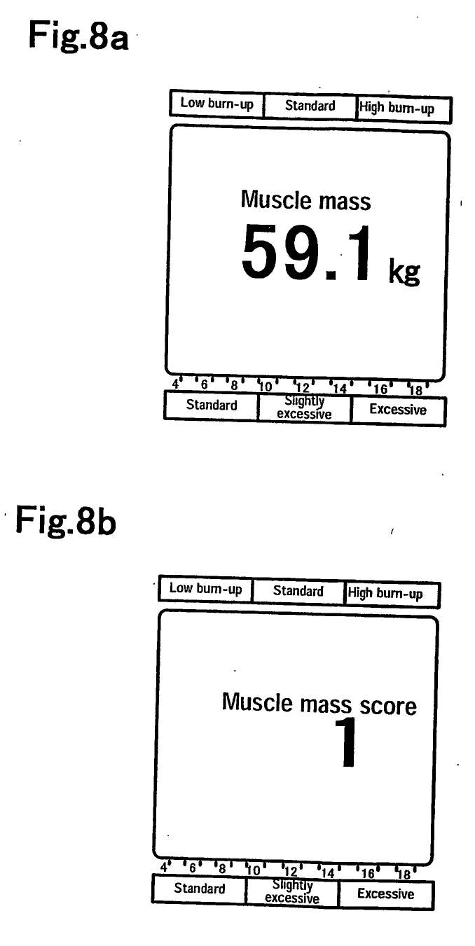

If the result of Step 19 is determined as “No”, it is determined whether or not the pressed key is the muscle key 15 in Step 22. If the pressed key is the muscle key 15, the result of Step 22 is determined as “Yes”, and the display shown in FIGS. 8a and 8b is performed in Step 23. First, as shown in FIG. 8a, the muscle mass of the overall body (e.g., 59.1 kg) calculated by a method according to Japanese Patent Application No. 2003-146842 is displayed for 3 seconds. Then, as shown in FIG. 8b, a muscle mass score “1” is displayed for 3 seconds. The muscle mass and the muscle mass score are alternately displayed until the power is turned off by the automatic power-off timer. If the muscle key 15 is pressed again while the muscle mass data is being displayed, the result of Step 24 is determined as “Yes”. Accordingly, the process returns to Step 14 and the main display items, the weight and the body fat ratio, are displayed again.

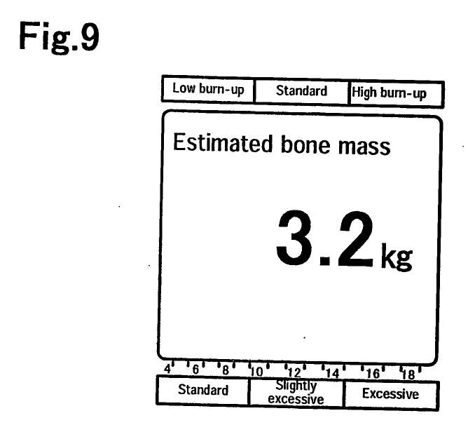

If it is determined that the pressed key is the bone mass key (that is, the down key) 12 in Step 22, the display shown in FIG. 9 is performed in Step 25. In FIG. 9, the bone mass of the overall body (e.g., 3.2 kg) calculated by a method according to Japanese Laid-Open Patent Publication No. 2002-65679 is displayed until the power is turned off by the automatic power-off timer. If the bone mass key 12 is pressed again while the bone mass data is being displayed, the result of Step 26 is determined as “Yes”. Accordingly, the process returns to Step 14 and the main display items, the weight and the body fat ratio, are displayed again.

In Steps 17, 20, 23, and 25, the secondary display items are continuously displayed until the automatic power-off timer runs out. However, if a key for displaying another secondary display item is pressed during the display, the processor portion 20 starts interrupt handling and the process returns to a point between Steps 15 and 16. Accordingly, the corresponding display item is displayed. For example, if the visceral fat key 14 on the input portion 6 is pressed while the basal metabolism data is being displayed as shown in FIG. 6 in Step 17, the processor portion 20 starts interrupt handling and the process proceeds to Step 20 from Step 19. Accordingly, the value corresponding to 1/10 of the visceral fat area and the bar graph shown in FIG. 7 are displayed. At this time, the automatic power-off timer is re-set to 30 seconds. The interrupt handling for displaying the secondary item is performed similarly for any of the basal metabolism data, the visceral fat data, the muscle mass data, and the bone mass data.

If the pressed key is neither the setting key 10 nor any of the personal keys 7a to 7d in Step 8, it is determined whether or not the pressed key is the guest key 11 in Step 27. If the pressed key is the guest key 11, the process proceeds to Step 28 and the personal data is registered, similar to Step 5. The personal data registered in Step 28 is displayed in Step 29, and then the process proceeds to Step 11, where the measurement is performed similarly to the case in which one of the personal keys 7a to 7d is pressed.

When the guest key 11 is pressed and the personal data is registered, this data is only memorized until the power is turned off at the end of the measurement and is cleared as the power is turned off. More specifically, when the guest key 11 is used, the personal data must be input each time the measurement is performed.

Next, if it is determined that the pressed key is the weight only/OFF key 8 in Step 27, the process proceeds to Step 30 and “00 kg” is displayed as the weight, similar to Step 11. After “00 kg” is displayed, the subject gets on the platform 2 of the body composition analyzer 1 and weight measurement is performed in Step 31. In this case, only the weight is measured and the power is turned off when the automatic power-off timer runs out.

1. A body composition analyzer that measures bioelectric impedance, comprising:
a display for displaying basic measurement results, and for displaying any one of a plurality of additional measurement results when the basic measurement results are not displayed;
a plurality of controls, each control corresponding to one of the additional measurement results; and
a processor configured for causing the display to display the basic measurement results, and further configured for causing the display to switch from displaying the basic measurement results to displaying a first one of the additional measurement results and not the basic measurement results, responsive to operation by a user of the control corresponding to the first one of the additional measurement results.

2. The body composition analyzer according to claim 1, wherein the basic measurement results comprise at least a body fat ratio, and the additional measurement results comprise basal metabolism data, visceral fat data, muscle mass data, and bone mass data.

3. The body composition analyzer according to claim 1, wherein the processor is configured to cause the display to display a second one of the additional measurement results responsive to operation by the user of the control corresponding to the second one of the additional measurement results when the first one of the additional measurement results is being displayed.

4. The body composition analyzer according to claim 2, wherein the basal metabolism data includes a basal metabolism rate and a result of an evaluation of the basal metabolism rate.

5. The body composition analyzer according to claim 4, wherein the basal metabolism rate is calculated using a fat free mass and the reciprocal of the subject's age.

6. The body composition analyzer according to one of claims 4 and 5, wherein the basal metabolism rate is evaluated by comparing the measured basal metabolism rate and a standard basal metabolism for the subject's age.

7. The body composition analyzer according to claim 2, wherein the visceral fat data includes a level of visceral fat area and a result of an evaluation of the visceral fat area.

8. The body composition analyzer according to claim 2, wherein the muscle mass data includes a muscle mass and a result of an evaluation of the muscle mass.

9. The body composition analyzer according to claim 8, wherein the muscle mass is evaluated on the basis of the muscle mass relative to the user's height.

10. The body composition analyzer according to claim 9, wherein the bone mass data is obtained using a lean body mass.

11. The body composition analyzer according to one of claims 1 to 3, further comprising a memory, wherein the processor is configured to calculate one of the measurement results based on reference information stored in the memory.

12. (canceled)

13. The body composition analyzer according to claim 1, wherein the controls comprise keys respectively corresponding to the additional measurement results.

14. The body composition analyzer according to claim 13, wherein the keys respectively correspond to at least basal metabolism, visceral fat, muscle mass and bone mass.

15. The body composition analyzer according to claim 1, wherein the processor is further configured for causing the display to switch from displaying the first one of the additional measurement results to displaying the basic measurement results, responsive to a second sequential operation by the user of the control corresponding to the first one of the additional measurement results.

16. The body composition analyzer according to claim 3, wherein the processor is further configured for causing the display to switch from displaying the second one of the additional measurement results to displaying the basic measurement results, responsive to a second sequential operation by the user of the control corresponding to the second one of the additional measurement results.

17. The body composition analyzer according to claim 1, wherein the basic measurement results comprise two separate measurement results, and wherein the processor is further configured for causing the display to alternately display each of the two separate measurement results when it is displaying the basic measurement results.

18. The body composition analyzer according to claim 2, wherein the basic measurement results further comprise a weight.

19. The body composition analyzer according to claim 18, wherein the processor is further configured for causing the display to alternately display the weight and the body fat ratio when it is displaying the basic measurement results.
